(19) United States
(12) Patent Application Publication
(10) Pub. No.: US 2009/0155200 A1
(43) Pub. Date: Jun. 18, 2009

(54) Methods of promoting cartilage healing or cartilage integration

(76) Inventor: Gregory D. Jay, Norfolk, MA (US)

Correspondence Address: CLARK & ELBING LLP, 101 FEDERAL STREET, BOSTON, MA, 02110, US

(21) Appl. No.: 11/587,115

(22) PCT Filed: Apr. 20, 2005
(86) PCT No.: PCT/US05/13372

§ 371 Date: Oct. 20, 2006

Related U.S. Application Data
(60) Provisional application No. 60/563,593, filed on Apr. 20, 2004.

(51) Int. Cl. A61K 38/20; A61K 31/77; A61K 39/395; A61K 38/48; A61K 31/711; A61K 31/737; A61K 31/015; A61K 31/713; A61P 19/04

(52) U.S. Cl. 424/78.3; 424/85.2; 424/133.1; 424/139.1; 424/94.64; 424/94.65; 424/94.66; 514/44; 514/54; 514/763

(57) ABSTRACT

The present invention provides methods of promoting cartilage healing or integration that include reducing the effective concentration of lubricin found in the extracellular matrix that is in contact with cartilaginous tissue.

### BACKGROUND OF THE INVENTION

The present invention relates to the healing and/or repair of cartilaginous tissue. It is generally agreed that injured articular cartilage has a limited intrinsic repair capacity. Clinical observations and animal experiments indicate that even thin fissures in articular cartilage can persist for years without healing (Buckwalter and Mankin, Instructional Course Lectures, eds. W. D. Cannon, Rosemont, USA American Academy of Orthopaedic Surgeons 1998:487-504; Hunziker, Osteoarthritis Cartilage 10:432-463, 2001). The poor integrative repair capacity of opposing cartilage surfaces has been related to the limited number of chondrocytes that are capable of migrating or proliferating in the cartilage-cartilage gaps (Hunziker, Clin. Orthop. 367: S135-S146, 1999). The insufficient cell recruitment could be due to the fact that articular cartilage is avascular and chondrocytes are entrapped in their own extracellular matrix (Caplan et al., Clin. Orthop. 342:254-269, 1997) and/or to the large extent of cell death that occurs following cartilage incisions (Hunziker and Quinn, Orthop. Res. 46:185, 2000). The limited cellularity within purely cartilaginous wounds could also be explained by the anti-adhesive properties of the defect surface conferred by dermatan sulfate and other proteoglycans (Hunziker and Rosenberg, J. Bone Joint Surg. Am. 78:721-733, 1996). For example, decorin and biglycan are known to inhibit adhesion of cells to macromolecules, such as fibronectin, in the extracellular matrix (Lewandowska et al., J. Cell. Biol. 105:1443-1454, 1987; Mitani et al., Rheumatol. Int. 20:180-185, 2001; and Schmidt et al., J. Cell. Biol. 104:1683-1691, 1987). In this context, molecules locally present in synovial fluid, which provide lubrication of the articular surface, are also likely to play a role in cartilage-cartilage integration.

Lubricin, also known as proteoglycan 4 (PRG4), articular cartilage superficial zone protein (SZP), megakaryocyte stimulating factor precursor, or tribonectin (Ikegawa et al., Cytogenet. Cell. Genet. 90:291-297, 2000; Schumacher et al., Arch. Biochem. Biophys. 311:144-152, 1994; Jay and Cha, J. Rheumatol., 26:2454-2457, 1999; and Jay, WIPO Int. Pub. No. WO 00/64930) is a mucinous glycoprotein found in the synovial fluid (Swann et al., J. Biol. Chem. 256:5921-5925, 1981). Lubricin provides boundary lubrication of congruent articular surfaces under conditions of high contact pressure and near zero sliding speed (Jay et al., J. Orthop. Res. 19:677-87, 2001). These lubricating properties have also been demonstrated in vitro (Jay, Connect. Tissue Res. 28:71-88, 1992). Cells capable of synthesizing lubricin have been found in synovial tissue and within the superficial zone of articular cartilage within diarthrodial joints (Jay et al., J. Rheumatol. 27:594-600, 2000).

In U.S. patent application Ser. No. 09/780,718 is described a monoclonal antibody to lubricin (SZP). In U.S. patent application Ser. No. 09/780,718 are described methods for detecting lubricin (SZP) and diagnosing degenerative conditions using an antibody specific for lubricin. In U.S. patent application Ser. No. 10/038,694 are described methods of promoting lubrication between two juxtaposed biological surfaces using lubricin, or fragments thereof. In PCT Publication No. WO 00/64930 are described lubricin (tribonectin) analogs and methods for lubricating a mammalian joint.

In a recent report (Englert et al., Trans. Orthop. Res. 29:189, 2003), the reduction of integration of opposing cartilage surfaces by components in synovial fluid was described and it was suggested that this reduction in integration was, at least in part, lubricin (SZP) mediated. What is needed are methods for promoting the healing or integration of cartilaginous tissue that include reducing the effective concentration of lubricin in synovial fluid.

### SUMMARY OF THE INVENTION

Accordingly, in a first aspect, the present invention features a method of promoting the healing or integration of cartilaginous tissue in a mammal that includes treating a cell capable of synthesizing lubricin, such as, for example, a chondrocyte or a synovial fibroblast, with a compound that inhibits the post-translational glycosylation of lubricin, thereby reducing the effective concentration of lubricin in the extracellular matrix (ECM) that contacts the cartilaginous tissue.

In an embodiment, the compound is an inhibitor of a glycosyltransferase, such as, for example, N-acetylneuraminyltransferase, N-acetylgalactosaminyltransferase, galactosyltransferase, N-acetylglucosaminyltransferase, or mannosyltransferase. Examples of inhibitors include N-acetylglucosamineβ1→6N-acetylgalactosamineα-O-2-naphthol, N-acetylglucosamineβ1→6galactoseβ-O-2-naphthol, N-acetylglucosamineβ1→6mannoseα-O-2-naphthol, N-acetylglucosamineβ1→2mannoseα-O-2-naphthol; galactoseβ1→3N-acetylgalactosamineα-O-2-naphthol, and galactoseβ1→4N-acetylglucosamineβ-O-2-naphthol.

In another aspect, the invention features a method of promoting the healing or integration of cartilaginous tissue in a mammal that includes treating a lubricin-synthesizing cell of the mammal, such as, for example, a chondrocyte or a synovial fibroblast, with a molecule having an antisense first nucleic acid sequence of sufficient length to inhibit the synthesis of lubricin in the cell, wherein the first nucleic acid sequence is complementary to a fragment of a second nucleic acid sequence, or one that is substantially identical to it, that encodes lubricin, thereby reducing the effective concentration of lubricin in the ECM that contacts the cartilaginous tissue. Preferably, the second nucleic acid sequence is SEQ ID NO. 1, which is the nucleic acid sequence that encodes human lubricin.

In another aspect, the invention features a method of promoting the healing or integration of cartilaginous tissue in a mammal that includes treating a cell of the mammal that is capable of synthesizing lubricin with an agent having double stranded RNA (dsRNA) in an amount sufficient to inhibit the synthesis of lubricin in the cell, wherein the RNA agent hybridizes to a fragment of a second nucleic acid sequence that encodes lubricin, thereby reducing the effective concentration of lubricin in the ECM that contacts the cartilaginous tissue. Preferably, the second nucleic acid sequence is SEQ ID NO. 1.

In another aspect, the invention features a method of promoting the healing or integration of cartilaginous tissue in a mammal that includes treating a lubricin-synthesizing cell of the mammal with a cytokine, wherein the administration of the cytokine reduces the effective concentration of lubricin in the ECM that contacts the cartilaginous tissue. In one embodiment the cytokine down-regulates the expression of lubricin. In another embodiment, the cytokine up-regulates the expression of proteolytic enzymes, resulting in the proteolysis of lubricin in the ECM. Preferably, the cytokine is IL-1α.

In another aspect, the present invention features a method of promoting the healing or integration of cartilaginous tissue in a mammal, such as, for example, a human patient, that includes treating the extracellular matrix (ECM) that is in contact with the cartilaginous tissue with an antibody that binds to lubricin. In one example, the antibody is a monoclonal antibody. In another example, the antibody is a humanized antibody. In yet another example, the antibody is not glycosyated.

In another aspect, the invention features a method of promoting the healing of or integration of cartilaginous tissue in a mammal that includes treating the extracellular matrix that contacts the tissue with a surfactant, thereby reducing the effective concentration of lubricin in the ECM. In one embodiment, the surfactant is a poloxamer, such as, for example poloxamer 188 (Pluronic™ F68), poloxamer 237, poloxamer 338, poloxamer 407, or a mixture thereof. In another embodiment, the surfactant is a carbomer, such as, for example, Carbopol™ 941, Carbopol 940, Carbopol 934, Carbopol 956, Ultrez 10, ETD-2020, or a mixture thereof.

In yet another aspect, the invention features a method of promoting the healing or integration of cartilaginous tissue in a mammal that includes treating the ECM that contacts the tissue with a proteolytic enzyme, wherein the enzyme affects the proteolysis of lubricin, thereby reducing the effective concentration of lubricin in the ECM. Preferably, the enzyme is administered locally in vivo. Examples of suitable proteolytic enzymes include papain, trypsin, chymotrypsin, subtilisin, pepsin, elastase, bromelain, ficin, Protease A, Protease B, Protease D, pepsin, thermolysin, pronase, dipeptidyl peptidase IV, granzyme A, granzyme B, granzyme K, cathepsin B, cathepsin K, cathepsin L, cathepsin S, and pancreatin. Preferably, the proteolytic enzyme is elastase, cathepsin B, cathepsin K, cathepsin L, or cathepsin S.

For all methods of the present invention a particularly desirable cartilaginous tissue to be healed or integrated is articular cartilage.

### DEFINITIONS

By “extracellular matrix” or “ECM” is meant the region outside of metazoan cells. This region includes compounds attached to the plasma membrane, as well as dissolved substances attracted to the surface charge of the cells. In general, the ECM functions both to keep animal cells adhered together, and well as buffering them from their environment. In a particular context of the present invention, the term “extracellular matrix” includes synovial fluid that is in contact with cartilaginous tissue.

By “cartilaginous tissue” is meant that connective tissue that consists of cells (e.g., chondrocytes) and interstitial substance (e.g. fibers) and a ground substance (chondromucoid). Cartilaginous tissue exists in three types, elastic cartilage, fibrocartilage, and articular cartilage. The methods of the present invention, while not limited to, most directly apply to cartilaginous tissue that is articular, meaning that cartilage which covers the ends of bones and allows the distribution of compressive loads over the cross section of bones and provides a frictionless wear-resistant surface for joint movement.

By “operably linked” is meant that a nucleic acid molecule and one or more regulatory sequences (e.g., a promoter) are connected in such a way as to permit expression and/or secretion of the product (i.e., a polypeptide) of the nucleic acid molecule when the appropriate molecules (e.g., transcriptional activator proteins) are bound to the regulatory sequences.

By “promoter” is meant a nucleic acid sequence sufficient to direct transcription, wherein such elements may be located in the 5′ or 3′ regions of the native gene.

By “reduce effective concentration” is meant to alter the appearance of a substance as normally found in a biological system in a manner that one or more of the substance's properties are diminished. In one example, reducing effective concentration can be achieved by lowering the concentration of a substance from that which is normally found in healthy tissue or biological fluid. In another example, a substance's effective concentration can be reduced by altering the chemical makeup of the substance (e.g., by changing the functional groups contained in the substance) such that certain properties, including those not related to biological function, are diminished. For example, changing the groups contained in a substance in a manner that diminishes its hydrogen-bonding character reduces the effective concentration of the substance, even in those cases where one, several, or all aspects of its normal biological function are maintained. In yet another example, the effective concentration of a substance can be reduced by altering the environment that surrounds the substance, such as, for example, by adding a surfactant to the biological milieu that contains the substance, resulting in a diminishment of the substances structured interaction with other components of the milieu.

By “substantially identical” is meant a peptide or nucleic acid sequence exhibiting at least 75%, but preferably 85%, more preferably 90%, most preferably 95%, or even 99% identity to a reference peptide or nucleic acid sequence. For polypeptides, the length of comparison sequences will generally be at least 20 amino acids, preferably at least 30 amino acids, more preferably at least 40 amino acids, and most preferably 50 amino acids. For nucleic acids, the length of comparison sequences will generally be at least 60 nucleotides, preferably at least 90 nucleotides, and more preferably at least 120 nucleotides.

### BRIEF DESCRIPTION OF THE DRAWINGS

FIG. 1 is a graph showing force-displacement curves during push-out mechanical tests. (A) Profiles acquired using untreated composites displayed a typical peak, indicating the dislocation of the disk from the ring. (B) Profiles acquired using lubricin treated composites did not display a clear peak, indicating negligible adhesion strength.

FIG. 2 is a graph showing the adhesive strength of lubricin treated and control composites. According to this parameter, treatment with lubricin resulted in a 10-fold decrease of integrative repair capacity.

FIG. 3 are microscopic photographs (Safranin-O staining, 100×) showing representative fields of disk/ring cartilage interfaces. FIG. 3A is an example of sample treatment by lubricin resulting in a pattern of interrupted contact zones. FIG. 3B is an example of near-homogenous distribution of GAG at the disk/ring interface in a control sample.

FIG. 4 is the amino acid sequence of human lubricin.

FIG. 5 is the nucleic acid sequence that encodes human lubricin (SEQ ID NO. 1).

### DETAILED DESCRIPTION

Mechanisms of integration of cartilage-cartilage interfaces have been investigated using different in vitro model systems, in order to decouple the biological processes that modulate the repair from the complex loading patterns in synovial joints (Reindel et al., J. Orthop. Res., 13:751-760, 1995). In particular, an in vitro disk-ring composite model has been described (Obradovic et al., J. Orthop. Res. 19:1089-1097, 2001) that mimics aspects of clinical methods of chondral transplantation by measuring dislocation of a cartilage disk from a cartilage ring.

In the present invention, we hypothesize that lubricin, a lubricating protein physiologically present in the synovial fluid, reduces the integrative cartilage repair capacity, and use this disk-ring model system to verify this hypothesis. In particular, we found that treatment with lubricin reduced the adhesive strength by more than 10-fold. The details of this study are as follows:

### Generation and Culture of Disk/Ring Composites

Articular cartilage was harvested from the femoral-patellar grooves of 3-6 week old calves under aseptic conditions within 6 hours after death and rinsed thoroughly in phosphate buffered saline (PBS) supplemented with 100 U/ml penicillin and 100 μg/ml streptomycin. Top and bottom surfaces were removed from the cartilage explants, and surfaces cut flat at an approximate thickness of 2 mm. Discs (5.0 mm o.d.) and rings (10.0 mm o.d.) were cored out simultaneously from a cartilage explant using two concentric circular blades. Disks were immersed for 30 min in PBS, with or without 250 μg/ml of lubricin extracted and purified from pooled bovine synovial fluid, as previously described (Jay, Connect. Tissue. Res. 28:71-88, 1992). This concentration falls between the threshold and maximal values of 200 μg/ml and 250 μg/ml, respectively, previously determined for lubrication by lubricin in a glass-rubber model. Each disk was then placed back into the corresponding ring from which it was cored and the composites cultured for up to 6 weeks on an orbital shaker at 16 rpm in Dulbecco's Modified Eagle Medium (4.5 g/L glucose with nonessential amino acids) supplemented with 10% fetal bovine serum, 10 mM HEPES, 2 mM glutamine, 1 mM sodium pyruvate, 100 U/ml penicillin, 100 μg/ml streptomycin (all previous from Gibco; Grand Island, N.Y.), 0.1 mM ascorbic acid 2-phosphate (Sigma; Buchs, CH), and 1 U/ml insulin (Novo Nordisk A/S; Bagsvaerd, Denmark). At each change of medium (3 times/week) samples initially treated with lubricin were incubated for 5 min with 20 μl lubricin solution applied on the top surface of each construct. A total of 34 disk-ring composites were generated and cultured.

### Assessment of Disk/Ring Composites

After 6 weeks of culture, the contact area was measured for each composite as the percentage of the disk/ring interface which did not transmit light when analyzed using a light transmission microscope. Mechanical properties of the disk/ring interface were assessed from a push-out test in which a plunger displaced the disk from the cartilage ring using a custom-made mechanical testing device (Obradovic et al., J. Orthop. Res. 19:1089-1097, 2001). Displacement of the plunger at 0.50 mm/min was controlled by a computer activated micro-stepper motor, and the push-out force was measured by a load cell coupled to the plunger. For each specimen, load measurements were first recorded until the disk was fully displaced from the ring, then load measurements were repeated with the disk removed. The second set of data, which functioned as a baseline, was subtracted from the first data set to remove any contribution to the force measurements due to friction between the plunger and the cartilage ring. The adhesive strength was evaluated as the maximum force to failure per unit of interfacial area. The interfacial area was determined for each composite by measuring with an electronic caliper the thickness of the disk and the ring at four different locations. For each experimental group, 6 to 7 composites were tested mechanically. Values are presented as averages ±the standard error of the mean, and statistical differences among experimental groups were assessed using nonparametric Mann-Whitney tests and considered significant at values of p<0.05. Composites were also assessed histologically by Safranin-O stain of horizontal cross-sections.

### Results

After 6 weeks of culture, the contact area at the disk/ring interface reached an approximate average of 90% for both experimental groups. For one of the composites, following lubricin treatment, the disk was completely displaced from the surrounding ring, and was therefore not included in the data analysis.

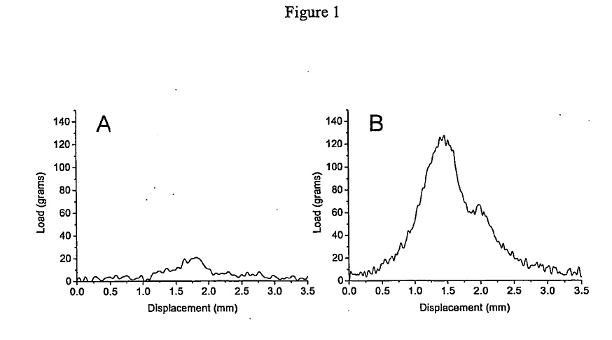

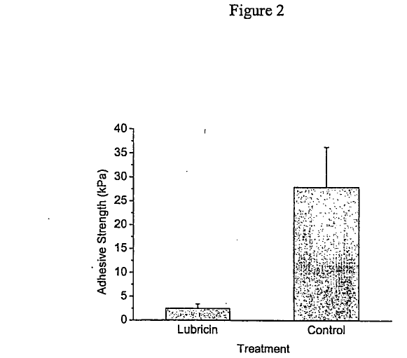

As shown in FIG. 1, mechanical testing resulted in characteristic force-displacement profiles, with forces increasing until a complete displacement of the disk occurred. As compared to lubricin-treated specimens, control composites displayed peaks in the force-displacement profiles which reached higher levels and were better defined. As shown in FIG. 2, the adhesive strength of control composites (28.7±8.3 kPa) was markedly and significantly higher (p=0.004) than in lubricin treated specimens (2.5±0.9 kPa).

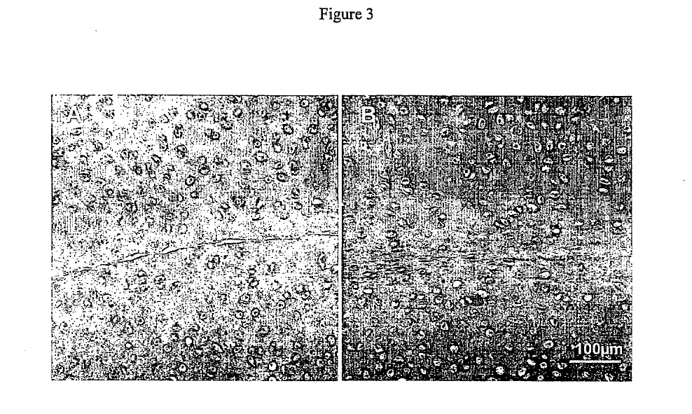

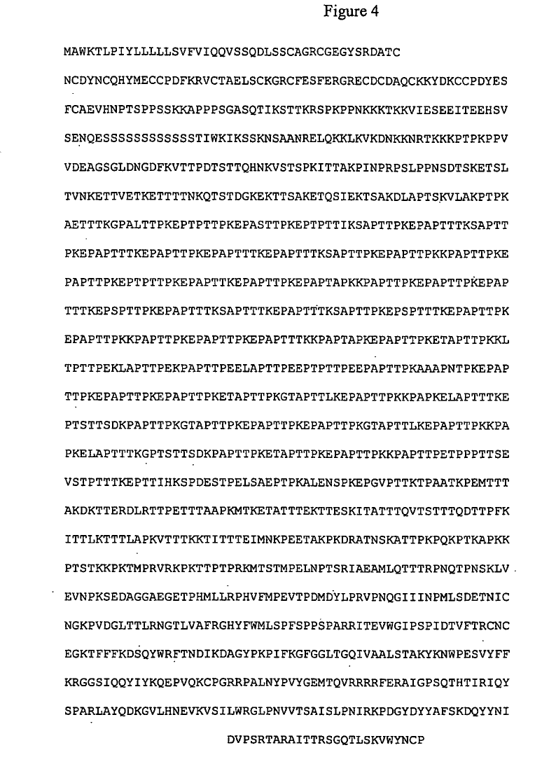

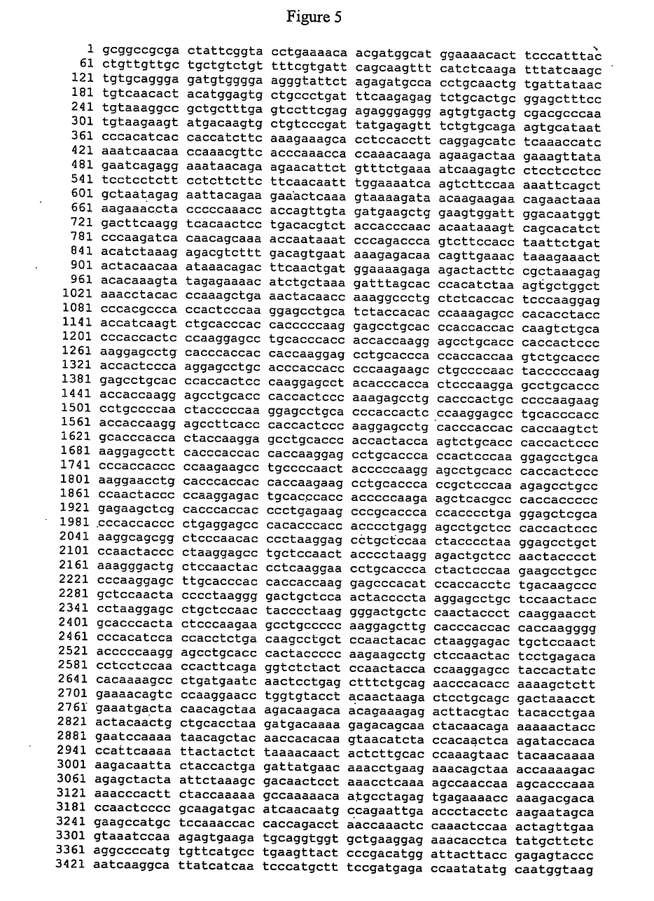

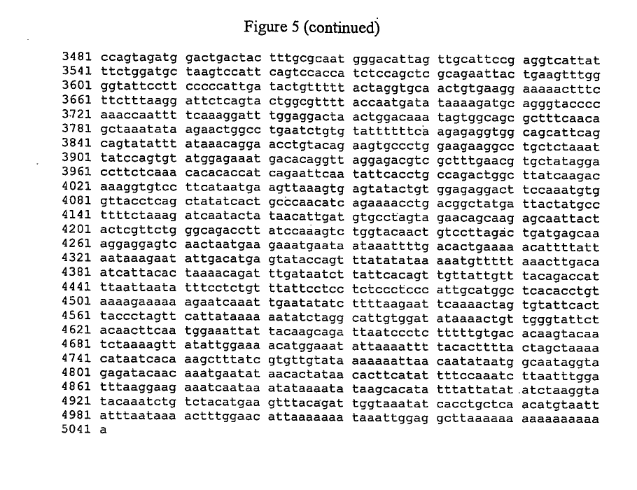

Histological analysis demonstrated a uniform and intense staining for Safranin-O of both disks and rings in composites treated and not treated with lubricin. In both groups the disk/ring interface could be identified in all explants. As shown in FIG. 3, lubricin-treated composites displayed a pattern of interrupted contact zones, whereas control composites had few interruptions.

### Lubricin in Synovial Fluid Inhibits Cartilage Integration

Lubricin has been proposed to be a key factor for joint lubrication. Homozygous knock-out mice lacking the orthologous gene PRG4, displayed significantly inferior joint lubrication as compared to the wild-type (Jay et al., Trans. Orthop. Res. 28:136, 2003). The boundary lubricating mechanism of lubricin has been related to the parallel orientation of lubricin molecules at the surface of articular cartilage, resulting in a repulsive hydration force if the distance between two opposing surfaces is less than 30 Å (Israelachvili, Intermolecular and surface forces with applications to colloidal and biological systems, Academic Press, New York, pp. 201-207, 1965; Jay, Connect. Tissue. Res. 28:71-88, 1992). In an oscillating glass-rubber model it has been shown that the lubricating properties of purified bovine lubricin are dose-dependent, such that the lubrication ability occurs only at concentrations higher than 200 μg/ml. In an example provided herein, a lubricin concentration of 250 μg/ml (i.e., that which is necessary for boundary lubrication) was repeatedly applied at the cartilage-cartilage interface and found to markedly reduce cartilage-cartilage integration in vitro.

The finding that lubricin reduces the integrative capacity of articular cartilage provides a plausible explanation for the limited ability of cartilage defects or even small cartilage fissures to heal. Described herein are methods of promoting cartilage integration or healing in a subject in need thereof that feature reducing the effective concentration of lubricin in the extracellular matrix that contacts cartilaginous tissue (e.g., articular cartilage) under healing or repair.

### Glycosyltransferase Inhibitors

Lubricin is a glycoprotein whose amino acid sequence contains approximately 28% threonine and serine residues which can be variously glycosylated with N-acetylneuraminic acid, galactosamine, and galactose, and to a small extent glucosamine and mannose. Lubricin's function is highly dependent upon this glycosylation. Therefore, inhibition of the post-translational process that produces lubricin in for example, chondrocytes or synovial fibroblasts, should dramatically alter its effective concentration in the synovial fluid milieu and improve cartilage integration or cartilage healing. Accordingly, one aspect of the present invention features a method of promoting the healing or integration of cartilaginous tissue in a mammal, such as, for example, a human patient, that includes treating cells that synthesize lubricin, such as, for example, those chondrocytes or synovial fibroblasts found in the ECM that contacts the cartilaginous tissue, with a compound that inhibits lubricin glycosylation. In one embodiment, the compound inhibits a glycosyltransferase enzyme, preferably N-acetylneuraminyltransferase, N-acetylgalactosaminyltransferase, galactosyltransferase, N-acetylglucosaminyltransferase, or mannosyltransferase.

Inhibitors of glycosyltransferases have been described by Hashimoto et al., J. Org. Chem. 62:1914-1915, 1997; Hashimoto et al., J. Synth. Org. Chem. Japan 55:325-333, 1997; Muller et al., Angewandte Chemie-Int. Ed. 37:2893-2897, 1998; Amann et al., Chemistry—A European Journal 4:1106-1115, 1998; Murray et al., Biochemistry 36:823-831, 1997; Kim et al., J. Am. Chem. Soc. 121:5829-5830, 1999; Schmidt et al., Bioorg. Med. Chem. 3:1747-1750, 1993; Miura et al., Bioorg. Med. Chem. 6:1481-1489, 1998; Palcic et al., J. Biol. Chem. 264:17174-17181, 1989; Kajihara et al., Carbohydr. Res. 247:179-193, 1993; Stults et al., Glycobiology 9:661-668, 1999; Lu et al., Bioorg. Med. Chem. 4:2011-2022, 1996; Lowary et al., Carbohydr. Res. 251:33-67, 1994; Khan et al., J. Biol. Chem. 268:2468-2473, 1993; Brown et al., Trends in Glycoscience and Glycotechnology 13:335-343, 2001; Neville et al., Biochem. J. 307:791-797, 1995; Kuan et al., J. Biol. Chem. 264:19271-19277, 1989; Sarkar et al., Proc. Natl. Acad. Sci. USA 92:3323-3327, 1995; and Sarkar et al., J. Biol. Chem. 272:25608-25616, 1997.

Esko et al., in U.S. Pat. No. 5,639,734, describe glycosyltransferase inhibitors that permeate cell membranes. These inhibitors consist of an aglycone moiety, such as, for example, naphthol, naphthalenemethane, indenol, a heterocyclic analog of indenol, a heterocyclic analog of naphthol, or a heterocyclic analog of naphthalenemethanol that is bonded to a sugar moiety, such as, for example, N-acetylneuraminic acid, galactose, N-acetylglucosamine, N-acetylgalactosamine, or mannose. Representative examples include: 1) N-acetylglucosamineβ1→6N-acetylgalactosamineα-X—R; (2) N-acetylglucosamineβ1→6 galactoseβ-X—R; (3) N-acetylglucosamineβ1→6mannoseα-X—R; (4) N-acetylglucosamineβ1→2mannoseα-X—R; (5) galactoseβ1→3N-acetylgalactosamineα-X—R; (6) galactoseβ1→4N-acetylglucosamineβ-X—R; (7) fucoseα1→4N-acetylglucosamineβ-X—R; and (8) fucoseα1→3N-acetylglucosamineβ-X—R, wherein X is a bridging atom selected from the group consisting of oxygen, sulfur, nitrogen and carbon; and wherein R is an aglycone selected from the group consisting of: naphthol, naphthalenemethane, indenol, a heterocyclic analog of indenol, a heterocyclic analog of naphthol, and a heterocyclic analog of naphthalenemethanol, wherein the aryl ring of the heterocyclic analog contains one or two nitrogen atoms that replace a methine (i.e., CH) moiety.

DeFrees in U.S. patent application Ser. No. 10/658,823, describes glycosyltransferase inhibitors that are based on the hydrophobic interactions between the carbohydrate portion of the enzyme substrates, or product, and the glycosyltransferase.

Proteolytic Enzymes The elimination of the lubricating activity of molecules of the synovial fluid by trypsin has been described (Jay and Cha, J. Rheumatol., 26:2454-2457, 1999), as well as the finding that cartilage treatment with trypsin may enhance its integration capacity by digesting cartilage proteoglycans (Obradovic et al., J. Orthop. Res. 19:1089-1097, 2001). Indeed, when rabbit patellar cartilage was implanted in a full-thickness articular cartilage defect, integration and remodeling were improved by pre-treatment with trypsin (Chen et al., Arch. Orthop. Trauma. Surg. 12.0:587-591, 2000).

Accordingly, in another aspect, the invention provides a method of promoting the healing or integration of cartilaginous tissue in a mammal, such as, for example, a human patient, that includes treating the extracellular matrix that is in contact with the cartilage with a proteolytic enzyme that contributes to the proteolysis of lubricin. In one embodiment, the proteolytic enzyme is selected from the group of proteases consisting of: papain, trypsin, chymotrypsin, subtilisin, pepsin, elastase, bromelain, ficin, Protease A, Protease B, Protease D, pepsin, thermolysin, pronase, dipeptidyl peptidase IV, cathepsin B, cathepsin K, cathepsin L, cathepsin S, and pancreatin. Preferably, the proteolytic enzyme is elastase, granzyme A, granzyme B, granzyme K, cathepsin B, cathepsin K, cathepsin L, or cathepsin S. In another embodiment, the proteolytic enzyme is administered locally in vivo.

### Cytokines

In another aspect, the present invention provides a method of promoting the healing of or integration of cartilaginous tissue in a mammal, such as, for example, a human patient, by treating cells that produce lubricin, such as, for example, chondrocytes or synovial fibroblasts, with a cytokine. In one embodiment, the cytokine down-regulates the expression of lubricin in the cell. In another embodiment, the cytokine up-regulates the expression of proteolytic enzyme that contributes to the proteolysis of lubricin. Desirably, the cytokine is IL-1α.

### Antisense DNA

In another aspect, the present invention provides a method of promoting the healing of or integration of cartilaginous tissue in a mammal, such as, for example, a human patient, by treating cells that produce lubricin, such as, for example, chondrocytes or synovial fibroblasts, with a molecule having an antisense nucleic acid sequence of sufficient length to inhibit the synthesis of lubricin the cell. The antisense sequence is complementary to a second nucleic acid sequence, or a sequence that is substantially identical to this second nucleic acid sequence, that mediates the synthesis of lubricin (e.g. antisense DNA), thereby inhibiting the gene expression of lubricin. In one embodiment, the antisense sequence is complementary to a fragment of the nucleic acid sequence that encodes lubricin. In another embodiment, the antisense sequence hybridizes to a promoter that is operably-linked to the genetic sequence encoding lubricin.

The nucleic acid sequences of the present invention or portions thereof can be inserted into a vector used to propagate the sequences in a cell. Such vectors are introduced into cells and the cells are propagated to produce multiple copies of the vector. A useful type of vector is an expression vector. Coding regions of the nucleic acid sequences of the present invention or fragments thereof can be inserted into an expression vector under conditions appropriate for expression of the sequences. Such vectors, are introduced into cells under conditions appropriate for expression. In a preferred embodiment, the cell is human.

The invention thus provides nucleic acid constructs which encode sequences complementary to a fragment of the nucleic acid sequence that is responsible for the synthesis of lubricin (e.g., SEQ ID NO. 1), various DNA vectors containing those constructs for use in transducing eukaryotic cells, cells transduced with the nucleic acids, fusion proteins encoded by the above nucleic acids, and target gene constructs.

Each of the nucleic acids of this invention may further contain an expression control sequence operably linked to the coding sequence and may be provided within a DNA vector, e.g., for use in transducing eukaryotic cells. Some or all of the nucleic acids of a given composition, including any optional nucleic acids, may be present within a single vector or may be apportioned between two or more vectors. In certain embodiments, the vector or vectors are viral vectors useful for producing recombinant viruses containing one or more of the nucleic acids. The recombinant nucleic acids may be provided as inserts within one or more recombinant viruses which may be used, for example, to transduce cells in vitro or cells present within an organism, including a human or non-human mammalian subject. For example, lubricin-related nucleic acids may be present within a single recombinant virus or within a set of recombinant viruses, each of which containing one or more of the set of recombinant nucleic acids. Viruses useful for such embodiments include any virus useful for gene transfer, including adenoviruses, adeno-associated viruses (AAV), retroviruses, hybrid adenovirus-AAV, herpes viruses, lenti viruses, etc. In specific embodiments, the recombinant nucleic acid containing the target gene is present in a first virus and one or more or the recombinant nucleic acids encoding the transcription regulatory protein(s) are present in one or more additional viruses. In such multiviral embodiments, a recombinant nucleic add encoding a fusion protein containing a bundling domain and a transcription activation domain, and optionally, a ligand binding domain, may be provided in the same recombinant virus as the target gene construct, or alternatively, on a third virus. It should be appreciated that non-viral approaches (naked DNA, liposomes or other lipid compositions, etc.) may be used to deliver nucleic acids of this invention to cells in a recipient subject.

The invention also provides methods for rendering a cell capable of regulated expression of a target gene which involves introducing into the cell one or more of the nucleic acids of this invention to yield engineered cells which can express the appropriate fusion protein(s) of this invention to regulate transcription of a target gene. The recombinant nucleic acid(s) may be introduced in viral or other form into cells maintained in vitro or into cells present within an organism. The resultant engineered cells and their progeny containing one or more of these recombinant nucleic acids or nucleic acid compositions of this invention may be used in: a variety of important applications, including human gene therapy, analogous veterinary applications, the creation of cellular or animal models (including transgenic applications) and assay applications. Such cells are useful, for example, in methods involving the addition of a ligand, preferably a cell permeant ligand, to the cells (or administration of the ligand to an organism containing the cells) to regulate expression of a target gene. Particularly important animal models include rodent (especially mouse and rat) and non-human primate models. In gene therapy applications, the cells will generally be human and the peptide sequence of each of the various domains present in the fusion proteins (with the possible exception of the bundling domain) will preferably be, or be derived from, a peptide sequence of human origin.

### RNAi

In another aspect, the present invention provides a method of promoting the healing of or integration of cartilaginous tissue in a mammal, such as, for example, a human patient, by treating chondrocytes or synovial fibroblast with an agent having a double stranded RNA (dsRNA) in an amount sufficient to inhibit the intracellular synthesis of lubricin, wherein the double stranded RNA hybridizes to a portion of a nucleic acid sequence that encodes lubricin.

RNA interference (RNAi) is a phenomenon describing double-stranded (ds)RNA-dependent gene specific posttranscriptional silencing. Although initial attempts to harness this phenomenon for experimental manipulation of mammalian cells were foiled by a robust and nonspecific antiviral defense mechanism activated in response to long dsRNA molecules, it was found that synthetic duplexes of 21 nucleotide RNAs could mediate gene specific RNAi in mammalian cells, without invoking generic antiviral defense mechanisms (see Elbashir et al., Nature 2001, 411:494-498; Caplen et al., Proc. Natl. Acad. Sci. 2001, 98:9742-9747. The use of small RNAs that have been chemically synthesized is one avenue that has produced promising results. Several groups have recently described the development of DNA-based vectors capable of generating such siRNA within cells. In general, these vectors result in intracellular transcription of short hairpin (sh)RNAs that are efficiently processed to form siRNAs (see, for example, Paddison et al., Nature 2004, 428:427-431; Paddison and Hannon, Curr. Opin. Mol. Ther. 2003 5:217-24; Paddison et al., Proc. Natl. Acad. Sci. 2002, 99:1443-1448; Paddison et al., Genes & Dev. 2002, 16:948-958; et al., Proc. Natl. Acad. Sci. 2002, 8:5515-5520; and Brummelkamp et al., Science 2002, 296:550-553.

### Surfactants

In another aspect, the present invention features a method of promoting the healing or integration of cartilaginous tissue in a mammal, such as, for example, a human patient, that includes treating the extracellular matrix (ECM) that is in contact with the cartilaginous tissue with a surfactant. In one example, when added to the ECM, the surfactants described herein reduce the effective concentration of lubricin by reducing its physical concentration. In another example, the added surfactant reduces the effective concentration of lubricin by interfering with the hydrogen bond interaction established between lubricin's glycosyl groups and other biomolecules in the ECM milieu.

Representative examples of long chain or high molecular weight (>1000) surfactants include gelatin, casein, lecithin (phosphatides), gum acacia, cholesterol, tragacanth, polyoxyethylene allyl ethers, e.g., macrogol ethers such as cetomacrogol 1000, polyoxyethylene castor oil derivatives, polyoxyethylene sorbitan fatty acid esters, e.g., the commercially available Tweens, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethylcellulose phthalate, microcrystalline cellulose, magnesium aluminum silicate, triethanolamine, polyvinyl alcohol, and polyvinylpyrrolidene (PVP). The low molecular weight (<1000) include stearic acid, benzalkonium chloride, calcium stearate, glycerol monostearate, cetostearyl alcohol, cetomacrogol emulsifying wax, and sorbitan esters. Most of these surface modifiers are known pharmaceutical excipients and are described in detail in Remington: The Science and Practice of Pharmacy (20th ed.), ed. A. R. Gennaro, Lippincott Williams & Wilkins, 2000, Philadelphia, or in Encyclopedia of Pharmaceutical Technology, eds. J. Swarbrick and J. C. Boylan, 2002, Marcel Dekker, New York.

Particularly preferred long chain surfactants include polyvinylpyrrolidone, tyloxapol, poloxamers such as Pluronic™ F68, F77, and F108, which are block copolymers of ethylene oxide and propylene oxide, and polyamines such as Tetronic™ 908 (also known as Poloxamine 908), which is a tetrafunctional block copolymer derived from sequential addition of propylene oxide and ethylene oxide to ethylenediamine, available from BASF, dextran, lecithin, dialkylesters of sodium sulfosuccinic acid, such as Aerosol OT, which is a dioctyl ester of sodium sulfosuccinic acid, available from American Cyanamid, Duponol P™, which is a sodium lauryl sulfate, available from DuPont, Triton X-200™, which is an alkyl aryl polyether sulfonate, available from Rohm and Haas, Tween™ 20 and Tween 80, which are polyoxyethylene sorbitan fatty acid esters, available from ICI Specialty Chemicals; Carbowax™ 3550 and 934, which are polyethylene glycols available from Union Carbide; Crodesta™ F-110, which is a mixture of sucrose stearate and sucrose distearate, available from Croda Inc., Crodesta™ SL-40, which is available from Croda, Inc., and SA9OHCO, which is C₁₈H₃₇—CH(CON(CH₃)CH₂(CHOH)₄CH₂OH)₂. Other useful surface modifiers include: decanoyl-N-methylglucamide; n-decyl-β-D-glucopyranoside; β-decyl-β-D-maltopyranoside; n-dodecyl-β-D-glucopyranoside; n-dodecyl-β-D-maltoside; heptanoyl-N-methylglucamide; n-heptyl-β-D-glucopyranoside; n-heptyl-β-D-thioglucoside; n-hexyl-β-D-glucopyranoside; nonanoyl-N-methylglucamide; n-nonyl-β-D-glucopyranoside; octanoyl-N-methylglucamide; n-octyl-β-D-glucopyranoside; octyl-β-D-thioglucopyranoside; and the like.

Another useful long chain surfactant is tyloxapol (a nonionic liquid polymer of the alkyl aryl polyether alcohol type; also known as superinone or triton). This surfactant is commercially available and/or can be prepared by techniques known in the art. Yet another surfactant p-isononylphenoxypoly (glycidol) also known as Olin-10G or Surfactant 10-G, is commercially available as 10G from Olin Chemicals, Stamford, Conn.

One preferred long chain surfactant is a block copolymer linked to at least one anionic group. The polymers contain at least one, and preferably two, three, four or more anionic groups per molecule. Preferred anionic groups include sulfate, sulfonate, phosphonate, phosphate and carboxylate groups. The anionic groups are covalently attached to the nonionic block copolymer. The nonionic sulfated polymeric surfactant has a molecular weight of 1,000-50,000, preferably 2,000-40,000, and more preferably 3,000-30,000. In preferred embodiments, the polymer comprises at least about 50%, and more preferably, at least about 60% by weight of hydrophilic units, e.g., alkylene oxide units. The reason for this is that the presence of a major weight proportion of hydrophilic units confers aqueous solubility to the polymer.

A preferred class of block copolymers useful as surface modifiers herein includes block copolymers of ethylene oxide and propylene oxide. These block copolymers are commercially available as Pluronics™. Specific examples of the block copolymers include F68, F77, F108, F127, and the like.

Another preferred class of block copolymers useful herein include tetrafunctional block copolymers derived from sequential addition of ethylene oxide and propylene oxide to ethylene diamine. These polymers, in an unsulfated form, are commercially available as Tetronics™.

Carbomers are also suitable as surfactants that can be added to the ECM in contact with cartilaginous tissue. Carbomers are high molecular weight network polymers consisting of acrylic acid backbones and small amounts of polyalkenyl polyether crosslinking agents. Co-monomers such as C₁₀-C₃₀ alkyl acrylates are sometimes used to hydrophobically modify homopolymer carbomers to improve their electrolyte tolerance. Water soluble or (dispersible) polymer molecules possess the unique capacity to greatly increase the viscosity of the liquid in which they are dissolved (dispersed), even when present at concentrations considered quite low. Examples of carbomers useful for the present invention are carbopol 941™, carbopol 940™, carbopol 934™, carbopol 956™, Ultrez 10™, and ETD-2020™, and are available from the BF Goodrich Company.

### Antibodies

In another aspect, the present invention features a method of promoting the healing or integration of cartilaginous tissue in a mammal, such as, for example, a human patient, that includes treating the extracellular matrix (ECM) that is in contact with the cartilaginous tissue with an antibody that binds to lubricin. In one example, the antibody is a monoclonal antibody. In another example, the antibody is a humanized antibody. In yet another example, the antibody is not glycosyated. Lubricin-binding antibodies are described in U.S. Pat. No. 6,720,156 and in U.S. application Ser. No. 09/780,718.

### Chondral Disorders

A further embodiment of any of the aspects of the present invention features a method for the treatment of cartilaginous tissue damaged by injury that includes reducing the effective concentration of lubricin in the ECM that contacts the cartilage. Generally, the injury is traumatic. More specifically, the injury treated is microdamage or blunt trauma, a chondral fracture, an osteochondral fracture, traumatic synovitis, or damage to tendons, menisci, or ligaments. Desirably, the cartilage is contained within a mammal, including humans, and the amount administered is a therapeutically effective amount. In a specific embodiment, the injury can be the result of excessive mechanical stress or other biomechanical instability resulting from a sports injury or obesity.

### Administration

The compounds of the invention can be administered systemically or locally. Methods in the art are known for formulating the agent according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York). In a non-limiting example of systemic administration, a sterile solution of the compound (0.01 to 5 mmoles of agent per 0.05 mL to 10 mL of diluent) is prepared and it is injected intravenously. In a non-limiting example of local administration, a sterile solution of compound (1 to 250 μmoles for non-catalytic agents, 1 to 250 mmoles for proteolytic enzymes in (0.05 mL to 2.5 mL of diluent) is injected into the joint. For therapy of the knee, the most common locations chosen for local injection are proximolateral to the patella, proximomedial to the patella, and into the intercondylar notch (when the knee is flexed). The patient is then asked to flex and extend their knee several times after the injection to help diffuse the material around the joint.

All publications and patents cited in this specification are hereby incorporated by reference herein as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of promoting healing or integration of cartilaginous tissue in a mammal comprising administering to said mammal a compound that inhibits the post-translational glycosylation of lubricin in a cell, thereby reducing the effective concentration of lubricin in the extracellular matrix that contacts cartilaginous tissue.

2. The method of claim 1, wherein said compound inhibits a glycosyltransferase enzyme.

3. The method of claim 2, wherein said glycosyltransferase is N-acetylneuraminyltransferase, N-acetylgalactosaminyltransferase, galactosyltransferase, N-acetylglucosaminyltransferase, or mannosyltransferase.

4. The method of claim 1, wherein said compound is N-acetylglucosamineβ1→6N-acetylgalactosamineα-O-2-naphthol; N-acetylglucosamineβ1→6 galactoseβ-O-2-naphthol; N-acetylglucosamineβ1→6mannoseα-O-2-naphthol; N-acetylglucosamineβ1→2mannoseα-O-2-naphthol; galactoseβ1→3N-acetylgalactosamineα-O-2-naphthol; or galactoseβ1→4N-acetylglucosamineβ-O-2-naphthol.

5. The method of claim 1, wherein said cell is a chondrocyte or a synovial fibroblast.

6-26. (canceled)

27. The method of claim 1, wherein said cartilaginous tissue is articular cartilage.

28. The method of claim 1, wherein said mammal is a human.

29. The method of claim 1, wherein said mammal is a horse.

30. A method for promoting healing or integration of cartilaginous tissue in a mammal comprising administering to said mammal a composition comprising an antisense first nucleic acid sequence of sufficient length to inhibit the synthesis of lubricin in a mammalian cell, wherein said first nucleic acid sequence is complementary to a portion of a second nucleic acid sequence, or one substantially identical to it, that encodes lubricin, thereby reducing the effective concentration of lubricin in extracellular matrix that contacts cartilaginous tissue.

31. The method of claim 30, wherein said nucleic acid sequence encoding lubricin is set forth in SEQ ID NO. 1

32. The method of claim 30, wherein said cell is a chondrocyte or a synovial fibroblast.

33. The method of claim 30, wherein said cartilaginous tissue is articular cartilage.

34. The method of claim 30, wherein said mammal is a human.

35. The method of claim 30, wherein said mammal is a horse.

36. A method for promoting healing or integration of cartilaginous tissue in a mammal comprising administering to said mammal a composition comprising double stranded RNA (dsRNA) in an amount sufficient to inhibit the synthesis of lubricin in a mammalian cell, wherein said agent hybridizes to a portion of a second nucleic acid sequence that encodes lubricin, thereby reducing the effective concentration of lubricin in extracellular matrix that contacts cartilaginous tissue.

37. The method of claim 36, wherein said cartilaginous tissue is articular cartilage.

38. The method of claim 36, wherein said mammal is a human.

39. The method of claim 36, wherein said mammal is a horse.

40. A method for promoting healing or integration of cartilaginous tissue in a mammal comprising administering to said mammal a composition comprising a cytokine capable of reducing the amount of lubricin synthesized by a mammalian cell or increasing the amount of a proteolytic enzyme synthesized by said cell, thereby reducing the effective concentration of lubricin in extracellular matrix that contacts cartilaginous tissue.

41. The method of claim 40, wherein said cytokine is IL-1α.

42. The method of claim 40, wherein said cell is a chondrocyte or a synovial fibroblast.

43. The method of claim 40, wherein said cartilaginous tissue is articular cartilage.

44. The method of claim 40, wherein said mammal is a human.

45. The method of claim 40, wherein said mammal is a horse.

46. A method for promoting healing or integration of cartilaginous tissue in a mammal comprising administering to said mammal a composition comprising a surfactant capable of reducing the effective concentration of lubricin in extracellular matrix that contacts said tissue.

47. The method of claim 46, wherein said surfactant is a carbomer or a poloxamer.

48. The method of claim 47, wherein said poloxamer is poloxamer 188 (Pluronic F68), poloxamer 237, poloxamer 338, poloxamer 407, or a mixture thereof.

49. The method of claim 47, wherein said carbomer is Carbopol 941, Carbopol 940, Carbopol 934, Carbopol 956, Ultrez 10, ETD-2020, or a mixture thereof.

50. The method of claim 46, wherein said cartilaginous tissue is articular cartilage.

51. The method of claim 46, wherein said mammal is a human.

52. The method of claim 46, wherein said mammal is a horse.

53. A method for promoting healing or integration of cartilaginous tissue in a mammal comprising administering to said mammal a composition comprising an antibody that binds to lubricin and is capable of reducing the effective concentration of lubricin in extracellular matrix (ECM) that contacts said tissue.

54. The method of claim 53, wherein said antibody is a monoclonal antibody.

55. The method of claim 53, wherein said antibody is humanized.

56. The method of claim 53, wherein said antibody is not glycosylated.

57. The method of claim 53, wherein said cartilaginous tissue is articular cartilage.

58. The method of claim 53, wherein said mammal is a human.

59. The method of claim 53, wherein said mammal is a horse.

60. A method for promoting healing or integration of cartilaginous tissue in a mammal comprising administering to said mammal a composition comprising a proteolytic enzyme capable of reducing the effective concentration of lubricin in extracellular matrix (ECM) that contacts said tissue, wherein said enzyme effects the proteolysis of lubricin.

61. The method of claim 60, wherein said enzyme is selected from the group consisting of: papain, trypsin, chymotrypsin, subtilisin, pepsin, elastase, bromelain, ficin, Protease A, Protease B, Protease D, pepsin, thermolysin, pronase, dipeptidyl peptidase IV, cathepsin B, cathepsin K, cathepsin L, cathepsin S, and pancreatin.

62. The method of claim 60, wherein said enzyme is selected from the group consisting of: elastase, granzyme A, granzyme B, granzyme K, cathepsin B, cathepsin K, cathepsin L, and cathepsin S.

63. The method of claim 60, wherein said cartilaginous tissue is articular cartilage.

64. The method of claim 60, wherein said mammal is a human.

65. The method of claim 60, wherein said mammal is a horse.


SEQUENCE LISTING (2 sequences)

SEQ ID NO: 1 - DNA, 5041 bp, Homo sapiens
gcggccgcgactattcggtacctgaaaacaacgatggcatggaaaacacttcccatttacctgttgttgctgctgtctgttttcgtgattcagcaagtttcatctcaagatttatcaagctgtgcagggagatgtggggaagggtattctagagatgccacctgcaactgtgattataactgtcaacactacatggagtgctgccctgatttcaagagagtctgcactgcggagctttcctgtaaaggccgctgctttgagtccttcgagagagggagggagtgtgactgcgacgcccaatgtaagaagtatgacaagtgctgtcccgattatgagagtttctgtgcagaagtgcataatcccacatcaccaccatcttcaaagaaagcacctccaccttcaggagcatctcaaaccatcaaatcaacaaccaaacgttcacccaaaccaccaaacaagaagaagactaagaaagttatagaatcagaggaaataacagaagaacattctgtttctgaaaatcaagagtcctcctcctcctcctcctcttcctcttcttcttcaacaatttggaaaatcaagtcttccaaaaattcagctgctaatagagaattacagaagaaactcaaagtaaaagataacaagaagaacagaactaaaaagaaacctacccccaaaccaccagttgtagatgaagctggaagtggattggacaatggtgacttcaaggtcacaactcctgacacgtctaccacccaacacaataaagtcagcacatctcccaagatcacaacagcaaaaccaataaatcccagacccagtcttccacctaattctgatacatctaaagagacgtctttgacagtgaataaagagacaacagttgaaactaaagaaactactacaacaaataaacagacttcaactgatggaaaagagaagactacttccgctaaagagacacaaagtatagagaaaacatctgctaaagatttagcacccacatctaaagtgctggctaaacctacacccaaagctgaaactacaaccaaaggccctgctctcaccactcccaaggagcccacgcccaccactcccaaggagcctgcatctaccacacccaaagagcccacacctaccaccatcaagtctgcacccaccacccccaaggagcctgcacccaccaccaccaagtctgcacccaccactcccaaggagcctgcacccaccaccaccaaggagcctgcacccaccactcccaaggagcctgcacccaccaccaccaaggagcctgcacccaccaccaccaagtctgcacccaccactcccaaggagcctgcacccaccacccccaagaagcctgccccaactacccccaaggagcctgcacccaccactcccaaggagcctacacccaccactcccaaggagcctgcacccaccaccaaggagcctgcacccaccactcccaaagagcctgcacccactgcccccaagaagcctgccccaactacccccaaggagcctgcacccaccactcccaaggagcctgcacccaccaccaccaaggagccttcacccaccactcccaaggagcctgcacccaccaccaccaagtctgcacccaccactaccaaggagcctgcacccaccactaccaagtctgcacccaccactcccaaggagccttcacccaccaccaccaaggagcctgcacccaccactcccaaggagcctgcacccaccacccccaagaagcctgccccaactacccccaaggagcctgcacccaccactcccaaggaacctgcacccaccaccaccaagaagcctgcacccaccgctcccaaagagcctgccccaactacccccaaggagactgcacccaccacccccaagaagctcacgcccaccacccccgagaagctcgcacccaccacccctgagaagcccgcacccaccacccctgaggagctcgcacccaccacccctgaggagcccacacccaccacccctgaggagcctgctcccaccactcccaaggcagcggctcccaacacccctaaggagcctgctccaactacccctaaggagcctgctccaactacccctaaggagcctgctccaactacccctaaggagactgctccaactacccctaaagggactgctccaactaccctcaaggaacctgcacccactactcccaagaagcctgcccccaaggagcttgcacccaccaccaccaaggagcccacatccaccacctctgacaagcccgctccaactacccctaaggggactgctccaactacccctaaggagcctgctccaactacccctaaggagcctgctccaactacccctaaggggactgctccaactaccctcaaggaacctgcacccactactcccaagaagcctgcccccaaggagcttgcacccaccaccaccaaggggcccacatccaccacctctgacaagcctgctccaactacacctaaggagactgctccaactacccccaaggagcctgcacccactacccccaagaagcctgctccaactactcctgagacacctcctccaaccacttcagaggtctctactccaactaccaccaaggagcctaccactatccacaaaagccctgatgaatcaactcctgagctttctgcagaacccacaccaaaagctcttgaaaacagtcccaaggaacctggtgtacctacaactaagactcctgcagcgactaaacctgaaatgactacaacagctaaagacaagacaacagaaagagacttacgtactacacctgaaactacaactgctgcacctaagatgacaaaagagacagcaactacaacagaaaaaactaccgaatccaaaataacagctacaaccacacaagtaacatctaccacaactcaagataccacaccattcaaaattactactcttaaaacaactactcttgcacccaaagtaactacaacaaaaaagacaattactaccactgagattatgaacaaacctgaagaaacagctaaaccaaaagacagagctactaattctaaagcgacaactcctaaacctcaaaagccaaccaaagcacccaaaaaacccacttctaccaaaaagccaaaaacaatgcctagagtgagaaaaccaaagacgacaccaactccccgcaagatgacatcaacaatgccagaattgaaccctacctcaagaatagcagaagccatgctccaaaccaccaccagacctaaccaaactccaaactccaaactagttgaagtaaatccaaagagtgaagatgcaggtggtgctgaaggagaaacacctcatatgcttctcaggccccatgtgttcatgcctgaagttactcccgacatggattacttaccgagagtacccaatcaaggcattatcatcaatcccatgctttccgatgagaccaatatatgcaatggtaagccagtagatggactgactactttgcgcaatgggacattagttgcattccgaggtcattatttctggatgctaagtccattcagtccaccatctccagctcgcagaattactgaagtttggggtattccttcccccattgatactgtttttactaggtgcaactgtgaaggaaaaactttcttctttaaggattctcagtactggcgttttaccaatgatataaaagatgcagggtaccccaaaccaattttcaaaggatttggaggactaactggacaaatagtggcagcgctttcaacagctaaatataagaactggcctgaatctgtgtattttttcaagagaggtggcagcattcagcagtatatttataaacaggaacctgtacagaagtgccctggaagaaggcctgctctaaattatccagtgtatggagaaatgacacaggttaggagacgtcgctttgaacgtgctataggaccttctcaaacacacaccatcagaattcaatattcacctgccagactggcttatcaagacaaaggtgtccttcataatgaagttaaagtgagtatactgtggagaggacttccaaatgtggttacctcagctatatcactgcccaacatcagaaaacctgacggctatgattactatgccttttctaaagatcaatactataacattgatgtgcctagtagaacagcaagagcaattactactcgttctgggcagaccttatccaaagtctggtacaactgtccttagactgatgagcaaaggaggagtcaactaatgaagaaatgaataataaattttgacactgaaaaacattttattaataaagaatattgacatgagtataccagtttatatataaaaatgtttttaaacttgacaatcattacactaaaacagatttgataatcttattcacagttgttattgtttacagaccatttaattaatatttcctctgtttattcctcctctccctcccattgcatggctcacacctgtaaaagaaaaaagaatcaaattgaatatatcttttaagaattcaaaactagtgtattcacttaccctagttcattataaaaaatatctaggcattgtggatataaaactgttgggtattctacaacttcaatggaaattattacaagcagattaatccctctttttgtgacacaagtacaatctaaaagttatattggaaaacatggaaatattaaaattttacacttttactagctaaaacataatcacaaagctttatcgtgttgtataaaaaaattaacaatataatggcaataggtagagatacaacaaatgaatataacactataacacttcatattttccaaatcttaatttggatttaaggaagaaatcaataaatataaaatataagcacatatttattatatatctaaggtatacaaatctgtctacatgaagtttacagattggtaaatatcacctgctcaacatgtaattatttaataaaactttggaacattaaaaaaataaattggaggcttaaaaaaaaaaaaaaaaa

SEQ ID NO: 2 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIWKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTAPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGEMTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP